(19) United States
(12) Patent Application Publication
(10) Pub. No.: US 2026/0334918 A1
(43) Pub. Date: Oct. 29, 2026

(54) DYNAMIC DETERMINATION OF IMAGING SEQUENCE COMPLETENESS

(71) Applicant: KONINKLIJKE PHILIPS N.V., EINDHOVEN (NL)

(72) Inventors: Payaal Kiritkumar Patel, Reading, MA (US); Christopher Edward Haverstock, Melrose, MA (US); Hyeon Woo lEE, Cambridge, MA (US); Goutam Ghoshal, South Grafton, MA (US); Mohsen Zahiri, Cambridge, MA (US); Martha Gail Grewe Wilson, Andover, MA (US); Joyce Grube, Cambridge, MA (US)

(21) Appl. No.: 19/484,695

(22) PCT Filed: May 8, 2024
(86) PCT No.: PCT/EP2024/062664

§ 371 Date: Nov. 14, 2025

Related U.S. Application Data
(60) Provisional application No. 63/466,531, filed on May 15, 2023.
(60) Provisional application No. 63/536,748, filed on Sep. 6, 2023.

(51) Int. Cl. A61B 8/00; A61B 8/08; G06V 10/98

(52) U.S. Cl. CPC A61B 8/463; A61B 8/085; A61B 8/467; A61B 8/54; G06V 10/993; G06V 2201/031

(57) ABSTRACT

A controller for an ultrasound system includes a user interface, a memory that stores instructions, and a processor that executes the instructions. When executed by the processor, the instructions cause the controller to: obtain a plurality of ultrasound images; sequentially process the plurality of ultrasound images to identify at least one anatomical structure in each of the plurality of ultrasound images; determine relative completeness of an imaging sequence for each of the at least one anatomical structure; generate a display on the user interface showing an indication of relative completeness of the imaging sequence for each of the at least one anatomical structure; and update the display on the user interface each time one of the plurality of ultrasound images is processed to reflect an updated status of the relative completeness of the imaging sequence for each of the at least one anatomical structure.

### GOVERNMENT INTEREST

This invention was made with United States government support awarded by the United States Department of Health and Human Services under the grant number HHS/ASPR/BARDA 75A50120C00097. The United States has certain rights in this invention.

### BACKGROUND

The ability to perform ultrasound scans is a learned and perishable skill. The perishability of the skill limits widespread use of ultrasound across the care continuum to the detriment of patients, medical professionals, and healthcare costs. Training medical professionals and maintaining their ultrasound scanning skill in point of care ultrasound (POCUS) is an outstanding challenge. In the context of emergency care, the Focused Assessment with Sonography for Trauma (FAST) ultrasound exam is one the ultrasound exams known to suffer from the most severe fluctuations in accuracy (60%-100%) depending on the medical professional executing the scan protocol. The accuracy of a FAST ultrasound exam may depend on the number of exams the medical professional has performed in the past and the time from their last exam. In other words, the FAST ultrasound exam is a skill that requires practice to develop and maintain and the skill level may decay with time. New users of FAST ultrasound exams may currently receive one on one training from trainers who can provide expert guidance, and/or from periodic (e.g., weekly) review of practice FAST ultrasound exams. Both methods have dependencies and do not allow users to improve performance independently in real-time, though independent and real-time training may be required to scale the use of FAST ultrasound.

When a patient arrives at a medical facility with a blunt trauma, the emergency room team and the trauma surgery team may have minutes to diagnose if the patient has internal bleeding with the FAST ultrasound exam and eventually computed tomography (CT) scans. Diagnosis of internal bleeding may lead to careful observation or life-saving treatment. Currently, the standard of care for a stable patient is to use computed tomography for the diagnosis of internal bleeding, but if the patient is unstable, the FAST ultrasound exam is used. The variability in skill of users is a major reason why FAST ultrasound exams are not always effective, and a poor, incomplete exam may delay life-saving treatment for the patient. As an example, when a FAST ultrasound exam is initially negative, inexperienced ultrasound users tend to not continue sweeping through all features comprehensively, which reduces the clinical interpretability of the exam.

### SUMMARY

According to an aspect of the present disclosure, a controller for an ultrasound system includes a user interface, a memory that stores instructions, and a processor that executes the instructions. When executed by the processor, the instructions cause the controller to: obtain a plurality of ultrasound images; sequentially process the plurality of ultrasound images to identify at least one anatomical structure in each of the plurality of ultrasound images; determine relative completeness of an imaging sequence for each of the at least one anatomical structure; generate a display on the user interface showing an indication of relative completeness of the imaging sequence for each of the at least one anatomical structure; and update the display on the user interface each time one of the plurality of ultrasound images is processed to reflect an updated status of the relative completeness of the imaging sequence for each of the at least one anatomical structure.

According to another aspect of the present disclosure, an ultrasound system includes a user interface; a memory that stores instructions; and a processor that executes the instructions. When executed by the processor, the instructions cause the ultrasound system to: obtain a plurality of ultrasound images; sequentially process the plurality of ultrasound images to identify at least one anatomical structure in each of the plurality of ultrasound images; determine relative completeness of an imaging sequence for each of the at least one anatomical structure; generate a display on the user interface showing an indication of relative completeness of the imaging sequence for each of the at least one anatomical structure; and update the display on the user interface each time one of the plurality of ultrasound images is processed to reflect an updated status of the relative completeness of the imaging sequence for each of the at least one anatomical structure.

According to another aspect of the present disclosure, a tangible, non-transitory computer-readable medium stores instructions. When executed by a processor, the instructions cause the processor to: obtain a plurality of ultrasound images; sequentially process the plurality of ultrasound images to identify at least one anatomical structure in each of the plurality of ultrasound images; determine relative completeness of an imaging sequence for each of the at least one anatomical structure; generate a display on the user interface showing an indication of relative completeness of the imaging sequence for each of the at least one anatomical structure; and update the display on the user interface each time one of the plurality of ultrasound images is processed to reflect an updated status of the relative completeness of the imaging sequence for each of the at least one anatomical structure.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 illustrates a system for dynamic determination of imaging sequence completeness, in accordance with a representative embodiment.

FIG. 2 illustrates another system for dynamic determination of imaging sequence completeness, in accordance with a representative embodiment.

FIG. 3 illustrates a user interface in dynamic determination of imaging sequence completeness, in accordance with a representative embodiment.

FIG. 4 illustrates another user interface in dynamic determination of imaging sequence completeness, in accordance with a representative embodiment.

FIG. 5 illustrates another user interface in dynamic determination of imaging sequence completeness, in accordance with a representative embodiment.

FIG. 6 illustrates a method for dynamic determination of imaging sequence completeness, in accordance with a representative embodiment.

FIG. 7 illustrates a computer system, on which a method for dynamic determination of imaging sequence completeness is implemented, in accordance with another representative embodiment.

### DETAILED DESCRIPTION

In the following detailed description, for the purposes of explanation and not limitation, representative embodiments disclosing specific details are set forth in order to provide a thorough understanding of embodiments according to the present teachings. However, other embodiments consistent with the present disclosure that depart from specific details disclosed herein remain within the scope of the appended claims. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. Definitions and explanations for terms herein are in addition to the technical and scientific meanings of the terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

As used in the specification and appended claims, the singular forms of terms ‘a’, ‘an’ and ‘the’ are intended to include both singular and plural forms, unless the context clearly dictates otherwise. Additionally, the terms “comprises”, and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to”, “coupled to”, or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below.

As described herein, a display, smart phone and/or another type of user interface may be used to show exam progress for some or all types of ultrasound exams. The indication may provide information of how much of the ultrasound exam has been completed for one or more anatomical structures, how much of the ultrasound exam is left to complete for one or more anatomical structures, and/or other types of indications of imaging sequence completeness.

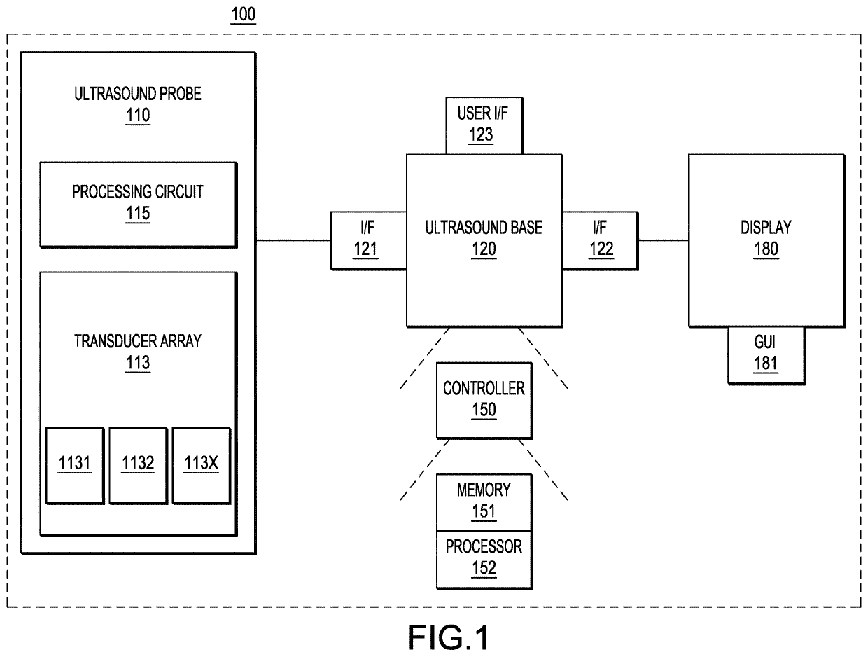

FIG. 1 illustrates a system 100 for dynamic determination of imaging sequence completeness, in accordance with a representative embodiment.

The system 100 in FIG. 1 is a system for dynamic determination of imaging sequence completeness and includes components that may be provided together or that may be distributed. The system 100 includes an ultrasound probe 110, an ultrasound base 120 and a display 180. The ultrasound probe 110 includes at least a transducer array 113 and a processing circuit 115. The transducer array 113 includes an array of individual transducer elements including a first transducer element 1131, a second transducer element 1132 through an Xth transducer element 113X. The transducer array 113 may include more than three transducer elements, such as dozens, hundreds or even thousands. The processing circuit 115 may include a combination of a memory that stores instructions and a processor that executes the instructions to, for example, process ultrasound imagery received via the transducer array 113. The processing circuit 115 may also or alternatively include circuit elements of an application-specific integrated circuit (ASIC). The ultrasound base 120 includes a first interface 121, a second interface 122, a third interface 123, and a controller 150. The third interface 123 is a user interface and may be used by users to interact with the ultrasound base 120 such as to enter instructions and receive output. In some alternative embodiments, the processing circuit 115 may also reside in the ultrasound base 120, such as when an ultrasound cart includes a passive circuit. Although not shown in FIG. 1, a cable may be provided as the connection between the ultrasound probe 110 and the ultrasound base 120. In FIG. 1, the controller 150 includes a memory 151 that stores instructions and a processor 152 that executes the instructions. The controller 150 in FIG. 1 also may include the third interface 123, and may also be considered to include the display 180 as a user interface in some embodiments.

The third interface 123 may allow a sonographer to provide instructions or other input to customize dynamic determination of imaging sequence completeness using the third interface 123 as a user interface. For example, a sonographer may be enabled to set up their own checklist and build customized conditions for completeness. The checklist may then be provided back to the sonographer via the display 180 or the third interface 123 as a user interface to use as a checklist of completed regions of interest as a history of what has been seen.

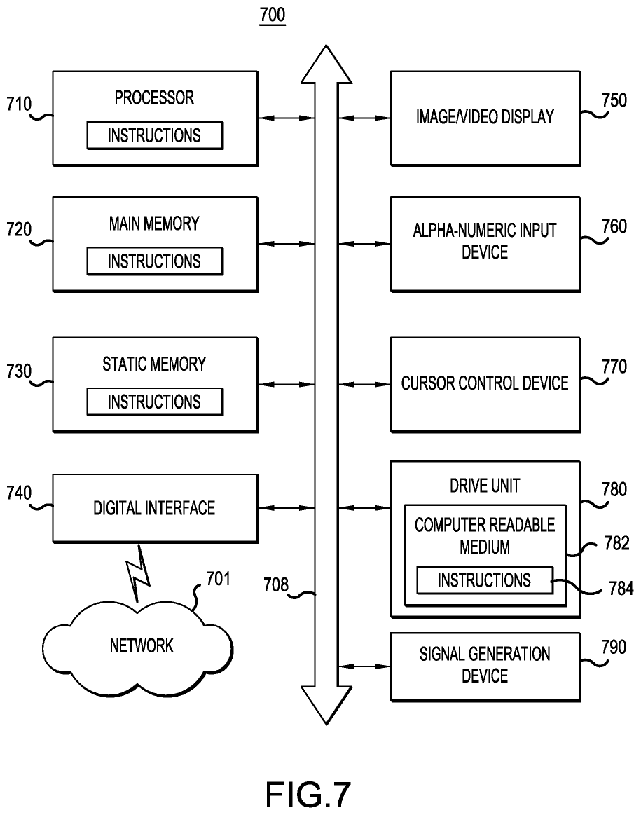

The controller 150 includes at least a memory 151 that stores instructions and a processor 152 that executes the instructions. A computer that can be used to implement the ultrasound base 120 is depicted in FIG. 7, though an ultrasound base 120 may include more or fewer elements than depicted in FIG. 1 or FIG. 7. In some embodiments, multiple different elements of the system 100 in FIG. 1 may include a controller such as the controller 150.

The display 180 may be local to the ultrasound base 120 or may be remotely connected to the ultrasound base 120. The display 180 may be connected to the controller 150 via a local wired interface such as an Ethernet cable or via a local wireless interface such as a Wi-Fi connection. The display 180 may be interfaced with other user input devices by which users can input instructions, including mouses, keyboards, thumbwheels and so on. The display 180 may be a monitor such as a computer monitor, a display on a mobile device, an augmented reality display, a television, an electronic whiteboard, or another screen configured to display electronic imagery. The display 180 may also include one or more input interface(s) such as those noted above that may connect to other elements or components, as well as an interactive touch screen configured to display prompts to users and collect touch input from users.

The controller 150 may perform some of the operations described herein directly and may implement other operations described herein indirectly. For example, the controller 150 may indirectly control operations such as by generating and transmitting content to be displayed on the display 180. The controller 150 may directly control other operations such as logical operations performed by the processor 152 executing instructions from the memory 151 based on input received from electronic elements and/or users via the interfaces. Accordingly, the processes implemented by the controller 150 when the processor 152 executes instructions from the memory 151 may include steps not directly performed by the controller 150.

When executed by the processor 152, the instructions cause the controller 150 to obtain ultrasound images via the transducer array 113, sequentially process the ultrasound images to identify at least one anatomical structure in each of the ultrasound images; and determine relative completeness of an imaging sequence for each of the at least one anatomical structure. The controller 150 generates data for display on the third interface 123 or the display 180 showing an indication of relative completeness of the imaging sequence for each of the at least one anatomical structure; and updates the display on the third interface 123 or the display 180 each time one of the plurality of ultrasound images is processed to reflect an updated status of the relative completeness of the imaging sequence for each of the at least one anatomical structure. The third interface 123 or the display 180 may therefore be used to provide a visualization of scan progress for quality assurance.

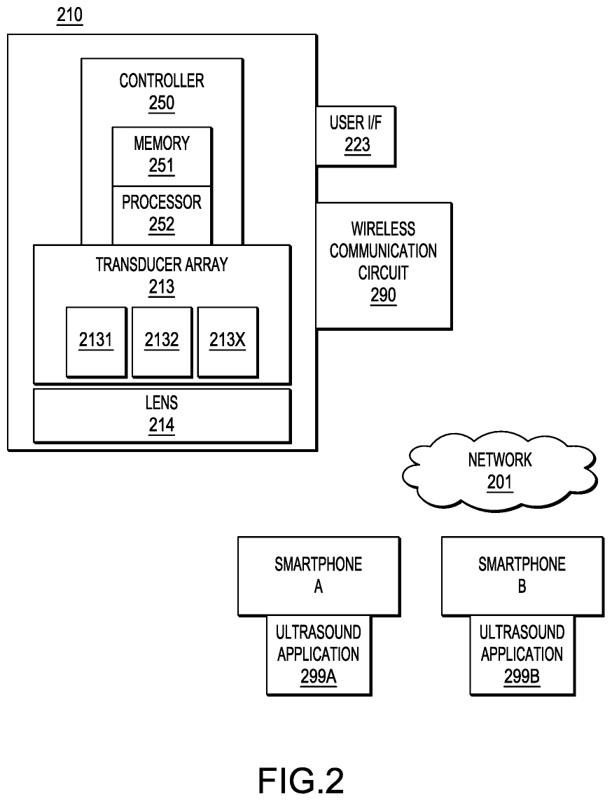

FIG. 2 illustrates another system for dynamic determination of imaging sequence completeness, in accordance with a representative embodiment.

The system 200 in FIG. 2 includes an ultrasound system 210. Additionally, while smartphone A and smartphone B are not necessarily considered part of the system 200 in FIG. 2, the ultrasound application 299A on smartphone A and the ultrasound application 299B on smartphone B may be considered part of the system 200 in FIG. 2. In some embodiments, smartphone A and/or smartphone B may be tablets or another form of mobile communication device rather than smartphones. The ultrasound system 210 includes a transducer array 213, a lens 214, a controller 250, a user interface 223 and a wireless communication circuit 290. In FIG. 2, the controller 250 includes a memory 251 that stores instructions and a processor 252 that executes the instructions. The controller 250 may also be considered to include the user interface 223 in some embodiments. In some embodiments, smartphone A and smartphone B may be considered part of the system 200, such as when issued by the same entity that owns or otherwise controls the ultrasound system 210. Smartphone A and smartphone B communicate with the ultrasound system 210 via network 201 and the wireless communication circuit 290. The network 201 may comprise one or more of a wireless local area network (LAN) such as a Wi-Fi network and/or a wired or wireless wide area network (WAN) such as a cellular network and/or the Internet. The ultrasound system 210 may comprise an ultrasound probe such as a portable ultrasound probe that coordinates ultrasound exams with the ultrasound application 299A and/or the ultrasound application 299B.

When executed by the processor 252, the instructions cause the controller 250 to obtain ultrasound images via the transducer array 213 and the lens 214, sequentially process the ultrasound images to identify at least one anatomical structure in each of the ultrasound images; and determine relative completeness of an imaging sequence for each of the at least one anatomical structure. The controller 250 may send the ultrasound images for display on a user interface of the smartphone A via the ultrasound application 299A and/or for display on a user interface of the smartphone B via the ultrasound application 299B. The display(s) on the user interface(s) of smartphone A and/or smartphone B may show an indication of relative completeness of the imaging sequence for each of the at least one anatomical structure. The controller 250 may send the indication of relative completeness to one or both of smartphone A and/or smartphone B for display with the ultrasound images. The controller 250 may also send updates for display on the user interface(s) of smartphone A and/or smartphone B each time one of the plurality of ultrasound images is processed to reflect an updated status of the relative completeness of the imaging sequence for each of the at least one anatomical structure. The user interface 223 or the smartphone A and/or the smartphone B may therefore be used to provide a visualization of scan progress for quality assurance.

Although not shown in FIG. 1 or FIG. 2, dynamic determination of imaging sequence completeness may be performed partially in a cloud context. For example, ultrasound images or metadata for ultrasound images may be sent via the network 201 to a cloud-based service that tracks completeness of imaging sequences for multiple distributed medical facilities and medical providers in real-time or near real-time.

The system 100 of FIG. 1 and the system 200 of FIG. 2 may be used to perform a FAST ultrasound exam. The system 100 of FIG. 1 or the system 200 of FIG. 2 may also be used to perform an extended FAST (eFAST) exam, or other types of exams like a full cardiac exam. The FAST ultrasound exam protocol has four main zones. Each of the four main zones has two main elements to use for judging whether the zone was investigated sufficiently. The first main element is the organs/features associated with the zone. The second main element is the regions of interest, which include spaces between organs in which fluid may collect. When the sonographer sets up preferences for completeness, the sonographer may be provided options to select one or more to apply to check for imaging sequence completeness. The options may include, for example, frame count, volume, area, connectivity, texture or any type of features that an image processing algorithm can qualify. The sonographer places the ultrasound probe 110 or the ultrasound system 210 on the region of interest on the patient's body. The display 180 or the smartphone A or the smartphone B may display an indicator of the quality of the ultrasound image. As the sonographer investigates the zone with the ultrasound probe 110 or the ultrasound system 210, the controller 150 or the controller 250 may recognize the degree to which each associated feature/organ was explored, such as by counting the number of frames in which the feature/organ appears. Using a frame count, an indication of relative completeness for an anatomical structure may be generated based on a count of ultrasound images in which the anatomical structure is present.

An example method for determining exam completeness may involve using the system 100 or the system 200 to receive real-time or near real-time ultrasound data, such as a cineloop of 2D images or 3D images. The controller 150 or the controller 250 may check quality of each ultrasound image using, for example, signal-to-noise ratio, resolution, structural similarity index, or a combination thereof. The controller 150 or the controller 250 may also check high quality images to determine a zone being imaged, such as whether a zone is a right upper quadrant (RUQ), left upper quadrant (LUQ), or Suprapubic (SP) zone being imaged in a FAST ultrasound exam. The controller 150 or the controller 250 may also detect the type of ultrasound exam, such as when not indicated by user input via the third interface 123 or the user interface 223. A checklist may be generated based on detecting the type of ultrasound exam, the sonographer identity, and/or any specific instructions input for the ultrasound exam. Completeness may then be determined sequentially after each ultrasound image is processed, and the display of the ultrasound images and the indications of completeness may be updated to show the ultrasound image and an indication of completeness.

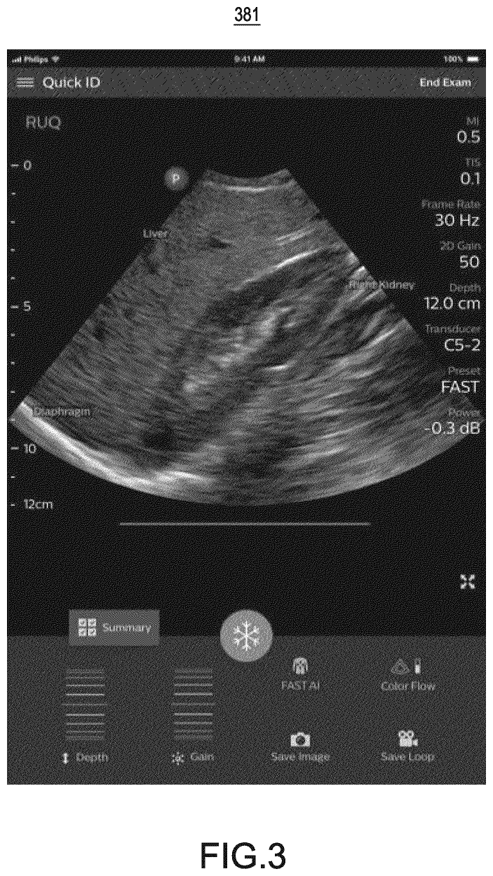

FIG. 3 illustrates a user interface in dynamic determination of imaging sequence completeness, in accordance with a representative embodiment.

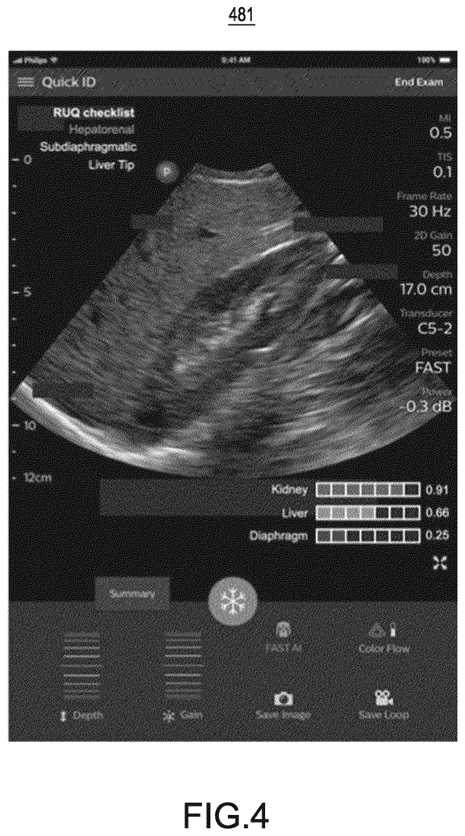

FIG. 3 shows an example of an image quality indicator on a user interface 381. When a medical professional places an ultrasound transducer on the region of interest on the patient's body, the ultrasound application will display an indicator of the quality of the image on the user interface 381. The image on the user interface 381 is an ultrasound image, and three different anatomical structures are shown and labeled. An eFAST. AI button is also shown for an extended FAST function for the lung as shown on the user interface 381. The three different anatomical structures are the right kidney, the liver, and the diaphragm. The labels for anatomical structures and/or regions of interest may be manually added by the sonographer, or detected by an image analysis algorithm or trained machine learning model. A summary screen may be generated upon detection or entry of the anatomical structure. A selectable link is provided for a user to select a summary, and once selected, the summary view shows the user interface of FIG. 4. A selectable link is also provided towards the bottom of the user interface 381 for the user to select fast AI (artificial intelligence), and once selected the resultant view shows a user interface 581 as in FIG.

FIG. 4 illustrates another user interface in dynamic determination of imaging sequence completeness, in accordance with a representative embodiment.

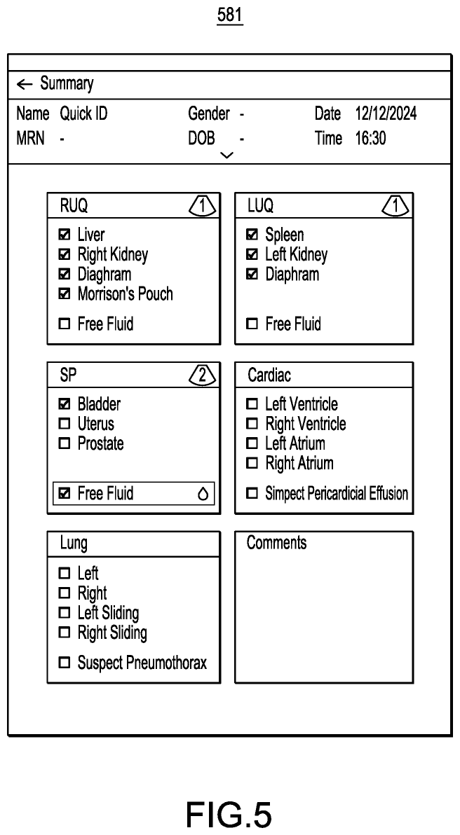

FIG. 4 shows a summary view as an image quality indicator on a user interface 481. The visual scale associated with each organ feature may start being populated as the user scans, and the visual scan may progress with each update as each new ultrasound image is captured. The scale may progress from red to yellow to green as the user fans through the organ, informing the user of the degree to which each feature was investigated. Three different status bars are shown on the user interface 481, one for each of the three different anatomical structures labeled on the user interface 381 in FIG. 3. The indication of relative completeness on the user interface 481 is shown as a color-coded feature, which varies a color for each anatomical structure based on relative completeness of imaging the anatomical structure. Accordingly, the indication of relative completeness on the user interface 481 is shown as a summary of relative completeness of imaging sequences for each of a plurality of anatomical structures. As shown, the status bar for the right kidney shows that ultrasound imaging of the right kidney is 91% complete. The status bar for the liver shows that ultrasound imaging of the liver is 66% complete. The status bar for the diaphragm shows that ultrasound imaging of the diaphragm is 25% complete. The status bars on the user interface 481 may be color coded, such as green when mostly complete, yellow when between 40% and 80% complete, and red when less than 40% complete. A green indication may indicate to a user that a corresponding anatomical structure in a region of interest has been sufficiently captured. A selectable link is provided for a user to select fast AI on the user interface 481, and once selected, the resultant view shows the user interface of FIG. 5.

FIG. 5 illustrates another user interface in dynamic determination of imaging sequence completeness, in accordance with a representative embodiment.

FIG. 5 shows a summary of information for anatomical structures on a user interface 581. The user interface 581 may be pre-populated as a results summary page of all scan data for accurate and easy documentation, and may be updated as the scan progresses. Completeness data generated by the system 100 or the system 200 may be saved with exam data so that the quality assurance and review committee of an ultrasound department can assess exam quality later.

Medical professionals may be provided with detailed feedback on technique, including the usability and completeness of the overall scan. Stated differently, the user interface 581 may show a medical professional relative incompleteness of features detected in an ultrasound scan. The anatomical structures for which information is shown on the user interface 581 include the three different anatomical structured labeled on the user interface 381 in FIG. 3. As shown, summary screens are populated for three different views of five possible views. The three different views for which information is populated on the user interface 581 include the RUQ view, the LUQ view, and the SP view. Accordingly, the indication of relative completeness on the user interface 581 is shown as a summary of relative completeness of imaging sequences for each of a plurality of anatomical structures. In FIG. 5, the RUQ list shows that the liver, right kidney, diaphragm and Morrison's pouch were all identified. The LUQ list shows that the spleen, left kidney and diaphragm were all identified. The SP list shows that only the bladder was identified, and highlights that free fluid was identified in the SP view. One of the most common causes of low exam quality is the incomplete investigation of a specific region within the zone, potentially missing fluid that may be in unexplored regions, so the indication of free fluid in a view is provided in the summary screens in FIG. 5. The cardiac list and the lung list are not populated on the user interface 581.

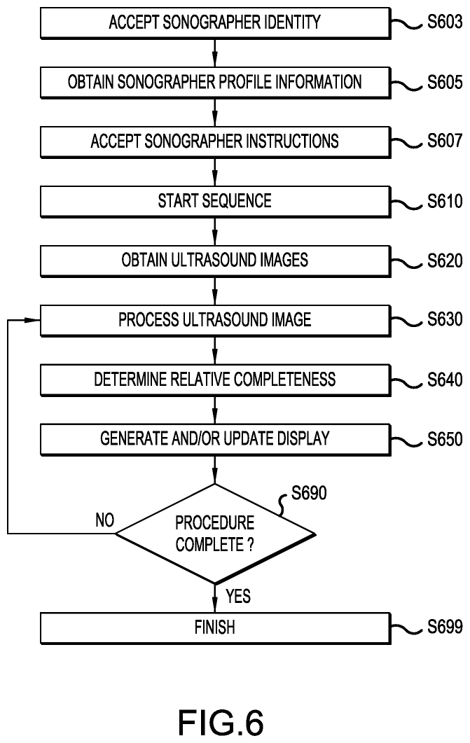

FIG. 6 illustrates a method for dynamic determination of imaging sequence completeness, in accordance with a representative embodiment.

The method of FIG. 6 may be performed by the system 100 including the controller 150 or by the system 200 including the controller 250.

At S603, a sonographer identity is accepted. The sonographer may be a physician, a physician assistant, a resident, or another type of medical professional. The sonographer identity may be input via the third interface 123 in FIG. 1, via the graphical user interface 181 in FIG. 1, via the user interface 223 in FIG. 2 or via either of smartphone A or smartphone B in FIG. 2.

At S605, sonographer profile information is obtained based on the sonographer identity. The sonographer profile information may be used for credentialing or a training check, for example. The sonographer profile information may be obtained from the memory 151 in FIG. 1 or from the memory 251 in FIG. 2. The sonographer profile information may be obtained at S605 to customize the dynamic determination of imaging sequence completeness for the sonographer, including skipping the dynamic determination of imaging sequence completeness in some circumstances such as when a sonographer is well experienced or simply does not wish to use dynamic determination of imaging sequence completeness.

At S607, sonographer instructions are accepted. Sonographer instructions may be input to the third interface 123 in FIG. 1, to the graphical user interface 181 in FIG. 1, to the user interface 223 in FIG. 2 or to either of smartphone A or smartphone B in FIG. 2. The sonographer instructions may be used to customize the dynamic determination of imaging sequence completeness, to specify the type of ultrasound exam being performed, to provide patient information for the patient being subjected to the ultrasound exam, or to otherwise enhance the usability of the dynamic determination of imaging sequence completeness.

At S610, a sequence is started. The sequence may comprise a sequence of ultrasound images to be captured in a scan. The sequence may be started by turning on an ultrasound probe and/or an ultrasound base. Alternatively, the sequence may be started by moving an ultrasound probe, or by placing the ultrasound probe against tissue or clothing of a patient. The sequence may also be started or restarted at S610 if the sonographer either lifts the ultrasound probe 110 or the ultrasound system 210, moves to another zone or saves a loop.

At S620, ultrasound images are obtained. For example, one or more ultrasound image(s) may be captured by the ultrasound probe 110 or the ultrasound system 210.

At S630, an ultrasound image is processed. The processing may involve identifying anatomical structures in the ultrasound image, as well as searching for features such as fluid that may indicate a health issue. For example, the processing at S630 may identify to the sonographer which anatomical zone the sonographer is scanning. The processing may be performed to assess quality of the ultrasound image, such as by analyzing the presence and quality of features present. The processing at S630 may also include a check on the quality of image captured, such as if an anatomical structure is only partially captured. Key features may be identified, and output based on the processing at S630.

At S640, relative completeness is determined. The relative completeness may be determined by adding a count of each anatomical structure in the ultrasound image to any previous count of the anatomical structure being present in a previous ultrasound image. The relative completeness may be qualified based on detection of key features and based on predefined conditions including any provided by the sonographer. Relative completeness may be indicated by how much of the ultrasound exam has been completed for one or more anatomical structures, how much of the ultrasound exam protocol is left for one or more anatomical structures, and/or other types of information. The sonographer may be enabled to set up their own checklist and build customized conditions for completeness. The sonographer may then be shown one or more indication of completeness as in FIG. 3, FIG. 4 and FIG. 5, indicating what has been seen and implicitly or explicitly indication what features the sonographer has left to scan. When the sonographer has provided a customized checklist, the customized checklist may be provided back to the sonographer as the checklist is completed during the scan.

Once the ultrasound probe 110 or the ultrasound system 210 is on the patient, the third interface 123 or display 180, or the user interface 223 or the smartphone A and/or smartphone B may provide guidance to the sonographer on what is remaining to capture in terms of clinically useful ultrasound imagery. The guidance may include a reminder of what view(s) are left to be captured, such as indications of regions suggested to explore in each zone and which regions have been completed. The guidance provided by dynamic determination of imaging sequence completeness may be particularly useful in scenarios when patients are subjected to FAST ultrasound exams. The output of the guidance and instruction algorithms may be automatically linked to a summary page as in FIG. 4 or FIG. 5, where the data can be viewed for completeness of the exam. The report generated as a summary page may also be used to facilitate the integration of the scan data into electronic medical records (EMR) systems, quality assurance (QA) systems, and billing.

Insofar as exam level quality of an ultrasound depends on the skill of the user in capturing all the views associated with the exam as well as capturing clinically useful images from each view, dynamic determination of imaging sequence completeness addresses challenges in point-of-care ultrasound sonographer skill development and decay.

A trained machine learning model may be used to process the ultrasound image at S630 and to determine relative completeness at S640. The trained machine learning model may use real-time clinical post-processing to track progress and predict relative completeness. The trained machine learning model may, for example, detect anatomical structures in ultrasound images. The system 100 or the system 200 may leverage the trained machine learning model to provide the sonographer with guidance on how well they have scanned necessary features within an ultrasound exam, such as within a FAST ultrasound exam.

At S650, a display is generated and/or updated. The display may show relative completeness of the imaging sequence for each of the anatomical structure(s) shown in the ultrasound image. In some embodiments, the display may show relative completeness of the imaging sequence for anatomical structure(s) not shown in the ultrasound image, but shown in previous ultrasound images.

The display at S650 may include a visual scale associated with each organ feature. The visual scale may start being populated as the sonographer starts scanning, and then progresses from red to yellow to green as the sonographer fans through the organ, informing the sonographer of the degree to which each feature was investigated. The display at S650 may also or alternatively include a checklist associated with important regions of interest. The visual scale, checklist or another indicator may turn green to inform the user when they have sufficiently captured the area.

An example of the logic for calculating the visual scale, checklist or other indicator may include a.) the indicator for the hepatorenal space within the RUQ lights up when the appropriate frames appear, b.) the indicator for the subdiaphragmatic space within the RUQ lights up when the appropriate frames appear, the indicator for the liver tip within RUQ lights up when the appropriate frames appear. Alternatively, a detected feature may be tracked using, for example, a bounding box, tracking from small to big to small to confirm coverage in the ultrasound scan for a particular feature/organ/area of interest.

The display at S650 may include one or more features for each region to indicate to the sonographer the level of completeness of the scan for each FAST ultrasound exam zone. The display may also explicitly or implicitly show what has already been seen to allow a less experienced sonographer to judge the quality of the scan they have completed and also to trigger the sonographer to further explore areas that may have been missed.

The completeness data generated by the system 100 or the system 100 may also be saved with exam data, and provided with the exam data for follow ups. A quality assurance and review committee of an ultrasound department may assess exam quality based on the exam data and completeness data, and provide sonographers with detailed feedback on technique as well as an assessment of usability of the ultrasound scan.

At S690, a determination is made as to whether the procedure is complete.

If the sequence is not complete (S690=No), the process returns to S630 and the next ultrasound image is processed. If the sequence is complete (S690=Yes), the process finishes at S699.

FIG. 7 illustrates a computer system, on which a method for dynamic determination of imaging sequence completeness is implemented, in accordance with another representative embodiment.

Referring to FIG. 7, the computer system 700 includes a set of software instructions that can be executed to cause the computer system 700 to perform any of the methods or computer-based functions disclosed herein. The computer system 700 may operate as a standalone device or may be connected, for example, using a network 701, to other computer systems or peripheral devices. In embodiments, a computer system 700 performs logical processing based on digital signals received via an analog-to-digital converter.

In a networked deployment, the computer system 700 operates in the capacity of a server or as a client user computer in a server-client user network environment, or as a peer computer system in a peer-to-peer (or distributed) network environment. The computer system 700 can also be implemented as or incorporated into various devices, such as a workstation that includes a controller, a base station, a smartphone, a standalone portable ultrasound system, a stationary computer, a mobile computer, a personal computer (PC), a laptop computer, a tablet computer, or any other machine capable of executing a set of software instructions (sequential or otherwise) that specify actions to be taken by that machine. The computer system 700 can be incorporated as or in a device that in turn is in an integrated system that includes additional devices. In an embodiment, the computer system 700 can be implemented using electronic devices that provide voice, video or data communication. Further, while the computer system 700 is illustrated in the singular, the term “system” shall also be taken to include any collection of systems or sub-systems that individually or jointly execute a set, or multiple sets, of software instructions to perform one or more computer functions.

As illustrated in FIG. 7, the computer system 700 includes a processor 710. The processor 710 may be considered a representative example of a processor of a controller and executes instructions to implement some or all aspects of methods and processes described herein. The processor 710 is tangible and non-transitory. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The processor 710 is an article of manufacture and/or a machine component. The processor 710 is configured to execute software instructions to perform functions as described in the various embodiments herein. The processor 710 may be a general-purpose processor or may be part of an application specific integrated circuit (ASIC). The processor 710 may also be a microprocessor, a microcomputer, a processor chip, a controller, a microcontroller, a digital signal processor (DSP), a state machine, or a programmable logic device. The processor 710 may also be a logical circuit, including a programmable gate array (PGA), such as a field programmable gate array (FPGA), or another type of circuit that includes discrete gate and/or transistor logic. The processor 710 may be a central processing unit (CPU), a graphics processing unit (GPU), or both. Additionally, any processor described herein may include multiple processors, parallel processors, or both. Multiple processors may be included in, or coupled to, a single device or multiple devices.

The term “processor” as used herein encompasses an electronic component able to execute a program or machine executable instruction. References to a computing device comprising “a processor” should be interpreted to include more than one processor or processing core, as in a multi-core processor. A processor may also refer to a collection of processors within a single computer system or distributed among multiple computer systems. The term computing device should also be interpreted to include a collection or network of computing devices each including a processor or processors. Programs have software instructions performed by one or multiple processors that may be within the same computing device or which may be distributed across multiple computing devices.

The computer system 700 further includes a main memory 720 and a static memory 730, where memories in the computer system 700 communicate with each other and the processor 710 via a bus 708. Either or both of the main memory 720 and the static memory 730 may be considered representative examples of a memory of a controller, and store instructions used to implement some or all aspects of methods and processes described herein. Memories described herein are tangible storage mediums for storing data and executable software instructions and are non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The main memory 720 and the static memory 730 are articles of manufacture and/or machine components. The main memory 720 and the static memory 730 are computer-readable mediums from which data and executable software instructions can be read by a computer (e.g., the processor 710). Each of the main memory 720 and the static memory 730 may be implemented as one or more of random access memory (RAM), read only memory (ROM), flash memory, electrically programmable read only memory (EPROM), electrically erasable programmable read-only memory (EEPROM), registers, a hard disk, a removable disk, tape, compact disk read only memory (CD-ROM), digital versatile disk (DVD), floppy disk, blu-ray disk, or any other form of storage medium known in the art. The memories may be volatile or non-volatile, secure and/or encrypted, unsecure and/or unencrypted.

“Memory” is an example of a computer-readable storage medium. Computer memory is any memory which is directly accessible to a processor. Examples of computer memory include, but are not limited to RAM memory, registers, and register files. References to “computer memory” or “memory” should be interpreted as possibly being multiple memories. The memory may for instance be multiple memories within the same computer system. The memory may also be multiple memories distributed amongst multiple computer systems or computing devices.

As shown, the computer system 700 further includes a video display unit 750, such as a liquid crystal display (LCD), an organic light emitting diode (OLED), a flat panel display, a solid-state display, or a cathode ray tube (CRT), for example. Additionally, the computer system 700 includes an input device 760, such as a keyboard/virtual keyboard or touch-sensitive input screen or speech input with speech recognition, and a cursor control device 770, such as a mouse or touch-sensitive input screen or pad. The computer system 700 also optionally includes a disk drive unit 780, a signal generation device 790, such as a speaker or remote control, and/or a network interface device 740.

In an embodiment, as depicted in FIG. 7, the disk drive unit 780 includes a computer-readable medium 782 in which one or more sets of software instructions 784 (software) are embedded. The sets of software instructions 784 are read from the computer-readable medium 782 to be executed by the processor 710. Further, the software instructions 784, when executed by the processor 710, perform one or more steps of the methods and processes as described herein. In an embodiment, the software instructions 784 reside all or in part within the main memory 720, the static memory 730 and/or the processor 710 during execution by the computer system 700. Further, the computer-readable medium 782 may include software instructions 784 or receive and execute software instructions 784 responsive to a propagated signal, so that a device connected to a network 701 communicates voice, video or data over the network 701. The software instructions 784 may be transmitted or received over the network 701 via the network interface device 740.

In an embodiment, dedicated hardware implementations, such as application-specific integrated circuits (ASICs), field programmable gate arrays (FPGAs), programmable logic arrays and other hardware components, are constructed to implement one or more of the methods described herein. One or more embodiments described herein may implement functions using two or more specific interconnected hardware modules or devices with related control and data signals that can be communicated between and through the modules. Accordingly, the present disclosure encompasses software, firmware, and hardware implementations. Nothing in the present application should be interpreted as being implemented or implementable solely with software and not hardware such as a tangible non-transitory processor and/or memory.

In accordance with various embodiments of the present disclosure, the methods described herein may be implemented using a hardware computer system that executes software programs. Further, in an exemplary, non-limited embodiment, implementations can include distributed processing, component/object distributed processing, and parallel processing. Virtual computer system processing may implement one or more of the methods or functionalities as described herein, and a processor described herein may be used to support a virtual processing environment.

Accordingly, dynamic determination of imaging sequence completeness enables display on a display, smart phone and/or another type of user interface of exam progress for some or all types of ultrasound exams. The indication may provide information of how much of the ultrasound exam has been completed for one or more anatomical structures, how much of the ultrasound exam is left to complete for one or more anatomical structures, and/or other types of indications of imaging sequence completeness. The display may include a checklist of progress of completeness, including progress for user-defined areas of interest within each zone. The indication of completeness may also be provided for each organ or feature, as well as by way of a summary for the overall ultrasound exam.

Although dynamic determination of imaging sequence completeness has been described with reference to several exemplary embodiments, it is understood that the words that have been used are words of description and illustration, rather than words of limitation. Changes may be made within the purview of the appended claims, as presently stated and as amended, without departing from the scope and spirit of dynamic determination of imaging sequence completeness in its aspects. Although dynamic determination of imaging sequence completeness has been described with reference to particular means, materials and embodiments, dynamic determination of imaging sequence completeness is not intended to be limited to the particulars disclosed; rather dynamic determination of imaging sequence completeness extends to all functionally equivalent structures, methods, and uses such as are within the scope of the appended claims.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept.

Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

We claim:

1. A controller for an ultrasound system, comprising:
a user interface;
a memory that stores instructions; and
a processor that executes the instructions, wherein, when executed by the processor, the instructions cause the controller to:
obtain a plurality of ultrasound images;
sequentially process the plurality of ultrasound images to identify at least one anatomical structure in each of the plurality of ultrasound images;
determine relative completeness of an imaging sequence for each of the at least one anatomical structure;
generate a display on the user interface showing an indication of relative completeness of the imaging sequence for each of the at least one anatomical structure; and
update the display on the user interface each time one of the plurality of ultrasound images is processed to reflect an updated status of the relative completeness of the imaging sequence for each of the at least one anatomical structure.

2. The controller of claim 1, wherein the plurality of ultrasound images are obtained in a focused assessment with sonography for trauma (FAST) exam.

3. The controller of claim 1, wherein the instructions further cause the controller to:
generate a display on the user interface showing which anatomical zone is being imaged when each of the plurality of ultrasound images is captured.

4. The controller of claim 1, wherein the instructions further cause the controller to:
generate a display on the user interface providing guidance on one or more anatomical zone left to be captured.

5. The controller of claim 1, wherein the indication of relative completeness is shown as a summary of relative completeness of imaging sequences for each of a plurality of anatomical structures.

6. The controller of claim 1, wherein the instructions further cause the controller to:
accept an indication of a sonographer using the ultrasound system to capture the plurality of ultrasound images; and
generate the display on the user interface based on the indication of the sonographer using the ultrasound system.

7. The controller of claim 6, wherein the display on the user interface based on the indication of the sonographer is customizable based on input from the sonographer.

8. The controller of claim 1, wherein the indication of relative completeness is shown as a checklist of completed regions of interest.

9. The controller of claim 1, wherein the indication of relative completeness is shown as a color-coded feature which varies a color for each anatomical structure based on relative completeness of imaging the anatomical structure.

10. The controller of claim 1, wherein the indication of relative completeness for an anatomical structure is generated based on a count of ultrasound images in which the anatomical structure is present.

11. An ultrasound system, comprising:
a user interface;
a memory that stores instructions; and
a processor that executes the instructions, wherein, when executed by the processor, the instructions cause the ultrasound system to:
obtain a plurality of ultrasound images;
sequentially process the plurality of ultrasound images to identify at least one anatomical structure in each of the plurality of ultrasound images;
determine relative completeness of an imaging sequence for each of the at least one anatomical structure;
generate a display on the user interface showing an indication of relative completeness of the imaging sequence for each of the at least one anatomical structure; and
update the display on the user interface each time one of the plurality of ultrasound images is processed to reflect an updated status of the relative completeness of the imaging sequence for each of the at least one anatomical structure.

12. The ultrasound system of claim 11, further comprising:
an ultrasound probe comprising a controller that includes the memory and the processor.

13. The ultrasound system of claim 11, wherein the instructions further cause the ultrasound system to:
generate a display on the user interface providing guidance on one or more anatomical zone left to be captured.

13. The ultrasound system of claim 11, wherein the indication of relative completeness is shown as a summary of relative completeness of imaging sequences for each of a plurality of anatomical structures.

14. The ultrasound system of claim 11, wherein the instructions further cause the ultrasound system to:
accept an indication of a sonographer using the ultrasound system to capture the plurality of ultrasound images; and
generate the display on the user interface based on the indication of the sonographer using the ultrasound system.

15. The ultrasound system of claim 14, wherein the display on the user interface based on the indication of the sonographer is customizable based on input from the sonographer.

16. The ultrasound system of claim 11, wherein the indication of relative completeness is shown as a color-coded feature which varies a color for each anatomical structure based on relative completeness of imaging the anatomical structure.

17. The ultrasound system of claim 11, wherein the indication of relative completeness for an anatomical structure is generated based on a count of ultrasound images in which the anatomical structure is present.

18. A tangible, non-transitory computer-readable medium that stores instructions, which when executed by a processor, cause the processor to:
obtain a plurality of ultrasound images;
sequentially process the plurality of ultrasound images to identify at least one anatomical structure in each of the plurality of ultrasound images;
determine relative completeness of an imaging sequence for each of the at least one anatomical structure;
generate a display on a user interface showing an indication of relative completeness of the imaging sequence for each of the at least one anatomical structure; and
update the display on the user interface each time one of the plurality of ultrasound images is processed to reflect an updated status of the relative completeness of the imaging sequence for each of the at least one anatomical structure.
